(19) United States
(12) Patent Application Publication
(10) Pub. No.: US 2019/0021881 A1
(43) Pub. Date: Jan. 24, 2019

(54) VOLUME-ADAPTABLE BREAST PROSTHESIS

(71) Applicant: Amoena Medizin-Orthopädie-Technik GmbH, Raubling (DE)

(72) Inventors: Nils STELTER, Frasdorf (DE); Helmut WILD, Stephanskirchen (DE)

(21) Appl. No.: 16/041,055

(22) Filed: Jul. 20, 2018

(30) Foreign Application Priority Data
Jul. 20, 2017 (DE) 10 2017 116 397.2

(51) Int. Cl. A61F 2/52

(52) U.S. Cl. CPC A61F 2/52; A61F 2002/5007

(57) ABSTRACT

The present disclosure relates to a breast prosthesis having an elastically deformable outer body whose outer surface is patterned on a shape of a breast and having a plastically deformable inner body whose inner surface can adapt to irregularities of tissue of a wearer. In an example of the breast prosthesis, the inner surface of the inner body has a three-dimensional structure and/or is provided with a textile that is respectively configured such that a ventilation of the tissue surface can be achieved, and an intermediate body having an intermediate chamber is arranged between the outer body and the inner body and is configured such that a volume of the intermediate chamber can be adapted by filling with a fluid.

### CROSS REFERENCE TO RELATED APPLICATION

This application claims priority to German Patent Application No. 10 2017 116 397.2 entitled “VOLUME-ADAPTABLE BREAST PROSTHESIS,” filed Jul. 20, 2017, the entire contents of which is hereby incorporated by reference in its entirety for all purposes.

### BACKGROUND

The present disclosure relates to a breast prosthesis having an elastically deformable body whose outer surface is patterned on the shape of the breast.

### TECHNICAL FIELD

Breast prostheses are used for cosmetic reasons after (partial) removals of the breast typically due to cancer. A high comfort in wear should in particular be achieved in addition to a shape and feel that comes as close as possible to the natural breast.

It is known from the prior art to provide the inner side of the prosthesis with a plastically deformable layer that adapts to the shape of the scar tissue. With these known prostheses, however, the problem can occur that the scar tissue was occlusively closed by this plastically deformable layer. This can result in heat accumulations and the formation of perspiration beneath the prosthesis, which is detrimental to the comfort in wear and can also result in skin reactions. Against this background, a breast prosthesis is presented in WO 2017/080664 A1 that has a plastically deformable inner body whose inner surface can adapt to the irregularities of the tissue, with the inner surface of the inner body having a three-dimensional structure and/or being provided with a textile that is respectively designed such that a ventilation of the tissue surface can be achieved. The inner side of the breast prosthesis can thus adapt to the shape of the scar tissue without preventing an air exchange.

The breast prosthesis of WO 2017/080664 A1, however, has the disadvantage that it can admittedly adapt to the shape of the scar tissue to a certain extent, but that an adaptation to the size of the remaining breast of the wearer cannot take place and a complete adaptation to the size of the tissue topography disposed beneath the prosthesis can frequently not take place.

It is the object of the present disclosure to provide a breast prosthesis whose inner side of the breast prosthesis can adapt to the shape of the scar tissue without preventing an exchange of air and which can furthermore be adapted individually in size to a patient.

Against this background, the present disclosure relates to a breast prosthesis having an elastically deformable outer body whose outer surface is patterned on a shape of a breast (e.g., a breast of a wearer of the breast prosthesis and/or a shape based on an average human breast shape) and having a plastically deformable inner body whose inner surface can adapt to irregularities of tissue (e.g., tissue of the wearer of the breast prosthesis, such as the tissue over which the breast prosthesis is placed when worn by the wearer), wherein the inner surface of the inner body has a three-dimensional structure and/or is provided with a textile that is respectively configured such that a ventilation of the tissue surface can be achieved. Provision is made in accordance with the present disclosure that an intermediate body having an intermediate chamber is arranged between the outer body and the inner body and a volume of the intermediate chamber can be adapted by filling with a fluid. In this way, the volume of the intermediate chamber may be filled with the fluid.

In an embodiment, the outer body and the intermediate body and the inner body contact one another directly.

In an embodiment, the breast prosthesis consists of the outer body, the intermediate body, the inner body, and the structure or the textile.

In an embodiment, the outer body is of shell shape. The intermediate body and the inner body may also be of shell shape.

In an embodiment, the outer body has a rubber-elastic compound enclosed in an outer chamber. The outer chamber may, for example, be formed by a film bag. The rubber-elastic compound may, for example, be a two-component silicone rubber compound.

In an embodiment, the inner body has a plastically deformable compound enclosed in an inner chamber. The inner chamber may, for example, also be formed by a film bag.

The plastically deformable compound may be a homogeneous or heterogeneous compound. The compound may have thixotropic properties. The compound may be a shear thinning compound having a yield point. Examples include low-crosslinked silicone gels, such as those having at least two components that may optionally further contain one or more additives. Suitable additives include phase change materials, expanded lightweight fillers, for example porous or gas-filled particles (non-expanded lightweight fillers may be added as part of the manufacturing process that expand due to temperature during the manufacturing process), thickening agents, aerosils, or thixotropic agents. Examples of further suitable plastically deformable compounds are disclosed in EP 0 768 068 A1, EP 2 133 042 A1 and WO 2014/078021 A1.

In an example embodiment, the intermediate body is formed by a film bag, that is, that the intermediate chamber is bounded by a film bag.

In an example embodiment, one or more of the film bags are formed by two plastic films welded along the common periphery.

In an example embodiment, the film bags forming the outer body, the intermediate body, and the inner body are formed by four plastic films welded along the common periphery. The inner boundary film of the outer body then corresponds to the outer boundary film of the intermediate body and the inner boundary film of the intermediate body corresponds to the outer boundary film of the inner body.

In an embodiment, a closable passage opening is provided that extends from the intermediate chamber to the outer side of the breast prosthesis. The filling of the intermediate chamber can be carried out with a fluid using this passage opening. Gases, liquids, or gels, such as silicone gels, may be used as filler materials. The passage opening may be a channel or an aperture.

In an example embodiment, the passage opening is located at the side contour of the breast prosthesis. For example, the passage opening may be a channel that extends between two layers of the weld seam between the films that form the intermediate body.

In an example embodiment, the passage opening is located at the inner surface of the breast prosthesis.

In a plan view, the breast prosthesis may have an approximately drop-like design that is patterned on the shape of the breast. In examples of this case, the passage opening may be located at or in the region of one of the two limbs. This has an advantage that the prosthesis can be placed into a brassiere cup and can then be filled at the sternum side or at the armpit side.

In an embodiment, a valve is provided in the passage opening. Self-sealing valves such as a flutter valve or a duckbill valve are suitable. It is of advantage for the comfort in wear if the valve is flexible or elastic.

The three-dimensional structure or the textile may be plastically and/or elastically deformable. The three-dimensional structure or the textile may be shaped such that a ventilation of the skin is ensured.

In an embodiment, the three-dimensional structure is molded to the inner surface of the inner body. In another embodiment, the three-dimensional structure is worked into the inner surface of the inner body. For example, the three-dimensional structure may form an integral part of the plastic film that represents the inner boundary of the inner body. The three-dimensional structure may be molded to this form or may be worked into it.

In an embodiment, the three-dimensional structure is adhesively bonded or welded to the inner surface of the inner body.

In an embodiment, the three-dimensional structure is a structure composed of plastic. The plastic material may be the same material that is used for the plastic film that represents the inner boundary of the inner body. Alternatively, different plastic materials may be used for the film and for the structure.

In an embodiment, the three-dimensional structure comprises channels, webs, and/or nubs.

In an embodiment, the textile is adhesively bonded or welded to the inner surface of the inner body.

In another embodiment, the textile is attached to the inner surface of the inner body using a holding means. For example, the textile may be attached to the inner surface of the inner body using a hook and loop surface adhesively bonded or welded to the inner surface of the inner body.

In an embodiment, the textile is a three-dimensional textile having a thickness of, for example, greater than 2 mm. For example, the textile may be a three-dimensional textile having a thickness of greater than 5 mm. Examples include two-surface or multi-surface textiles such as spacer fabrics or knitted spacer fabrics.

In an embodiment, the textile has a surface structure such as ribs or nubs at at least the surface facing the body.

In an embodiment, the textile comprises or consists of natural fibers. Natural fibers can have advantages in skin compatibility and in low odor take-up. Examples of suitable natural fibers include cotton fibers and/or wool fibers.

In an embodiment, the textile comprises or consists of plastic fibers. Plastic fibers can have advantages in good perspiration removal and small surface roughness.

Combinations of natural fibers and plastic fibers are also conceivable and covered by the present disclosure.

The present disclosure further relates to a method of adapting a breast prosthesis in accordance with the present disclosure, wherein the volume of the breast prosthesis is adapted by filling the intermediate chamber with a fluid. The filling may, for example, take place while the breast prosthesis is received in a brassiere cup and is worn by the patient. The filling may, for example, take place at the sternum side or at the armpit side. Gases, liquids, or gels, in particular silicone gels, are suitable as filler materials. The filling may take place by applying a filling apparatus to the outer side of the passage opening. Manual or electric pump apparatus are, for example, suitable as filling apparatus.

Further details and advantages result from the following embodiment discussed with reference to the Figures.

### BRIEF DESCRIPTION OF THE FIGURES

FIG. 1 shows a sectional view of a breast prosthesis in accordance with the present disclosure.

FIG. 2 shows a plan view of the breast prosthesis in accordance with the present disclosure. The figures are approximately to scale, although other relative dimensions may be used if desired.

### DETAILED DESCRIPTION

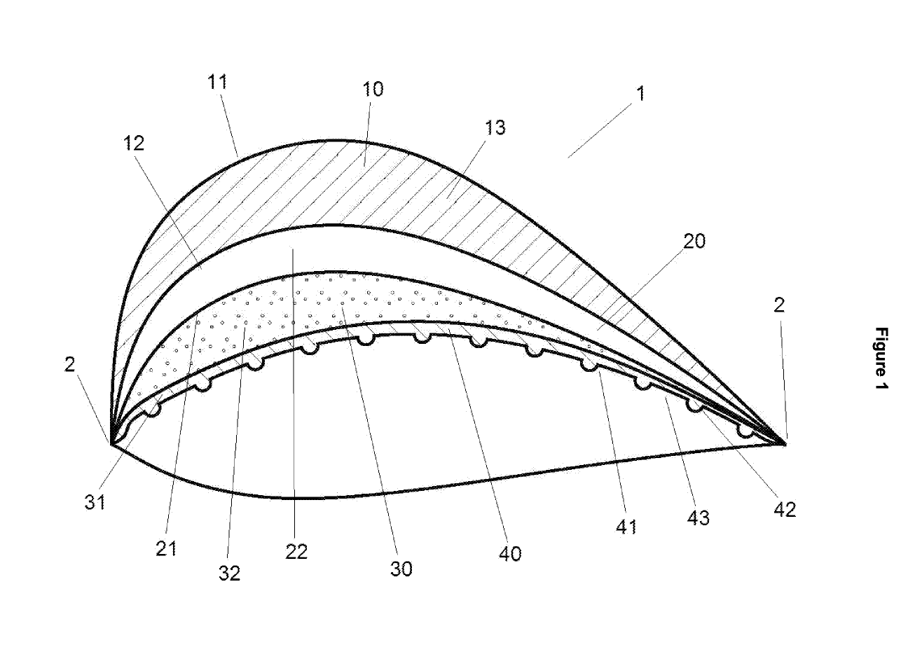

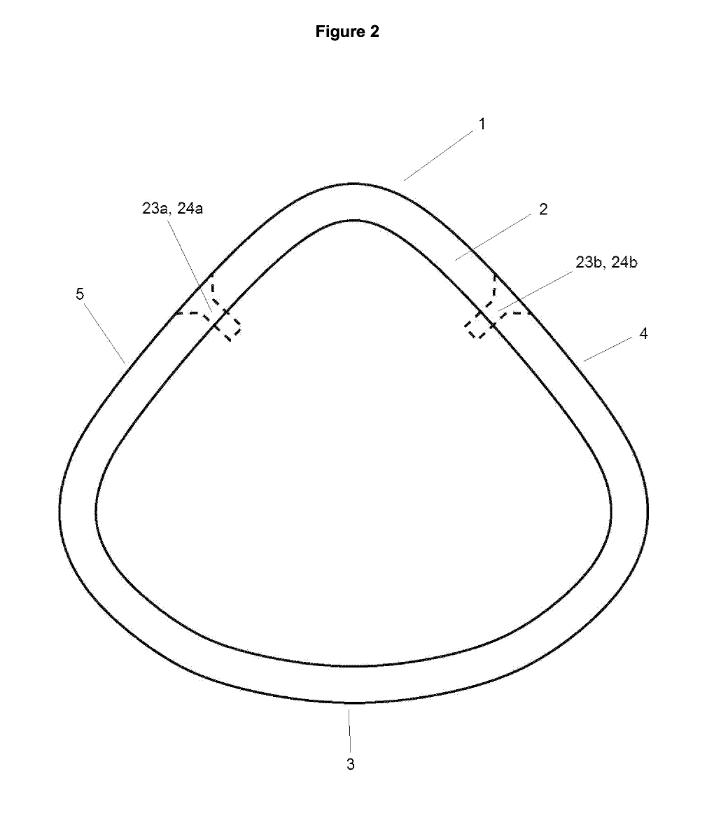

An embodiment of a breast prosthesis in accordance with the present disclosure that is generally marked by the reference numeral 1 is shown in FIGS. 1 and 2.

The outer side of the breast prosthesis is formed by a shell-shaped outer body 10 that is elastically deformable. The inner side of the breast prosthesis is formed by an inner body 30 likewise of shell shape. The bodies 10 and 30 are congruent, e.g., the bodies 10 and 30 have the same area and shape in a plan view. An expandable intermediate body 20 that is likewise of shell shape is arranged over the full area between the outer body 10 and the inner body 30.

The outer body 10 comprises two plastic films welded along the peripheral margin, namely an outer film 11 and an inner film 12 that form a chamber 13 in which a rubber elastic two-component silicone rubber compound is located.

The intermediate body 20 is formed by welding a further plastic film 21 to the inner side of the outer body 10.

The inner body 30 is formed by welding yet a further plastic film 31 to the inner side of the intermediate body 20 and by filling the interposed chamber 33 with a filler material. The filler material is a plastically deformable compound having thixotropic material properties which may be a low-crosslinked silicone gel of two different silicone components that further includes or contains phase change material, expanded lightweight fillers, thickening agents, aerosols, and thixotropic agents.

The plastic films 11, 12, 21, and 31 are all produced from an identical material. The plastic films 11, 12, 21, and 31 are connected along a common weld seam 2 in the marginal region of the prosthesis 1, with the weld seam not necessarily having to be disposed at the outer margin, but being able to be displaced, for example, inwardly peripherally by 10 mm-20 mm.

A structured plastic inlay 40 is adhesively bonded to the inner film 31 of the plastically deformable body 30. The adhesive surface of the inlay 40 is smooth to ensure a good adhesion to the inner film 31 of the inner body 30. The surface of the inlay 40 remote from the inner body 30 has a three-dimensional structure 41 which is a regular arrangement of nubs 42 and ventilation channels or folding channels 43.

An intermediate chamber 22 that can be filled with a fluid is formed in the interior of the intermediate body 20. Depending on the degree of filling, the thickness of the intermediate body increases by a specific amount. The volume of the breast prosthesis 1 can thus be adapted in consideration of the size of the remaining breast and of the progression of the scar tissue.

The breast prosthesis has a drop-like shape overall in a plan view, with the lower side being formed by the rounded portion 3 and with the two converging sides being formed by left and right limbs 4 and 5.

Two passage channels 23a and 23b are provided with an elastic flutter valve 24a and 24b each for filling the intermediate body 20. The channels 23 are arranged laterally at the breast prosthesis and are disposed between the films 12 and 21, that is, within the weld seam 2. They project radially outwardly starting from the intermediate chamber 20. They are arranged at the two limbs 4 and 5 of the breast prosthesis 1 so that the filling can take place selectively at the sternum side or at the armpit side after placing the prosthesis into a cup of the brassiere worn by the patient, independently of whether the prosthesis is worn at the left or right.

In the course of the filling of the intermediate chamber 22, a fluid, preferably a silicone gel, is urged by a pump from the outer side through the channel 23 and the self-closing flutter valve 24.

1. A breast prosthesis having an elastically deformable outer body whose outer surface is patterned on a shape of a breast and having a plastically deformable inner body whose inner surface can adapt to irregularities of tissue of a wearer, wherein the inner surface of the inner body has a three-dimensional structure and/or is provided with a textile that is respectively configured to enable ventilation of a surface of the tissue, wherein an intermediate body having an intermediate chamber is arranged between the outer body and the inner body and is configured such that a volume of the intermediate chamber is adapted by filling with a fluid.

2. The breast prosthesis in accordance with claim 1, wherein the outer body has a rubber elastic compound enclosed in an outer chamber formed by a film bag.

3. The breast prosthesis in accordance with claim 2, wherein the rubber elastic compound comprises a two-component silicone rubber compound.

4. The breast prosthesis in accordance with claim 1, wherein the inner body has a plastically deformable compound enclosed in an inner chamber formed by a film bag.

5. The breast prosthesis in accordance with claim 1, wherein the intermediate body is formed by a film bag.

6. The breast prosthesis in accordance with claim 1, further comprising a closable passage opening that extends from the intermediate chamber to an outer side of the breast prosthesis, wherein a valve is provided in the passage opening.

7. The breast prosthesis in accordance with claim 6, wherein the passage opening is located at a side contour or at the inner surface of the breast prosthesis.

8. The breast prosthesis in accordance with claim 6, wherein the valve comprises a self-closing valve.

9. The breast prosthesis in accordance with claim 6, wherein the passage opening includes a channel that extends between two layers of a weld seam between films that form the intermediate body.

10. The breast prosthesis in accordance with claim 1, wherein the three-dimensional structure is shaped to the inner surface of the inner body or is worked into the inner surface of the inner body.

11. The breast prosthesis in accordance with claim 1, wherein the three-dimensional structure is adhesively bonded or welded to the inner surface of the inner body.

12. The breast prosthesis in accordance with claim 1, wherein the three-dimensional structure is a structure of plastic; and/or wherein the three-dimensional structure comprises channels, webs, and/or nubs as structural elements.

13. The breast prosthesis in accordance with claim 1, wherein the textile is adhesively bonded or welded to the inner surface of the inner body.

14. The breast prosthesis in accordance with claim 1, wherein the textile is attached to the inner surface of the inner body using a holding mechanism.

15. The breast prosthesis in accordance with claim 14, wherein the holding mechanism comprises a hook and loop surface adhesively bonded or welded to the inner surface of the inner body.

16. A method of adapting a breast prosthesis in accordance with claim 1, wherein the volume of the breast prosthesis is adapted by filling the intermediate chamber with a fluid.

17. The method in accordance with claim 16, wherein the fluid is a silicone gel.

18. The method in accordance with claim 16, further comprising, in the course of the filling of the intermediate chamber, the fluid is urged by a pump from an outer side of the breast prosthesis through a channel and a valve to the intermediate chamber.

19. The method in accordance with claim 18, wherein the valve is a self-closing flutter valve.

20. The method in accordance with claim 18, wherein the filling of the intermediate chamber takes place selectively at a sternum side or at an armpit side of the breast prosthesis after placing the prosthesis into a cup of a brassiere.
